(19) United States
(12) Patent Application Publication
(10) Pub. No.: US 2013/0324944 A1
(43) Pub. Date: Dec. 5, 2013

(54) CLOSURE PLUG FOR A CATHETER

(71) Applicant: IBERICA DE REPRODUCCIÓN ASISTIDA, S.L., Centelles (ES)

(72) Inventor: Javier GIL PASCUAL, Centelles (ES)

(21) Appl. No.: 13/904,768

(22) Filed: May 29, 2013

(30) Foreign Application Priority Data
May 29, 2012 (ES) U201230581

(51) Int. Cl. A61M 39/20

(52) U.S. Cl. CPC A61M 39/20
USPC 604/256

(57) ABSTRACT

A closure plug for a catheter includes a hollow tubular main body with two open opposite ends, the first end for coupling to the final end of the catheter and the second end for coupling to a first open end of a tubular secondary body provided with a second closed end opposite the first end. The main body can include a support flange for supporting the secondary body and the plug can include securing means for securing the secondary body to the main body.

### TECHNICAL FIELD OF THE INVENTION

The present invention corresponds to the technical field of instruments used in medicine to introduce fluids into or extract fluids from the body.

### BACKGROUND OF THE INVENTION

Certain instruments such as cannulas or catheters are widely used today in medicine. These instruments are used for solving problems in which it is necessary to introduce fluids into or extract fluids from the body.

One of the specific fields of medicine in which cannulas and catheters are used is the artificial insemination of animals. Among other advantages, genetic improvement of the animals, reduction in the transmission of infectious diseases existing in the male or female genital organs, as well as an increase in fertility, are thereby achieved.

The technique which is most commonly used today consists of passing a catheter through the entire cervical canal. According to the method used, semen is deposited at the beginning of the cervix in the body of the uterus or by way of deep intrauterine insemination. In these last two cases, in addition to the catheter, it is necessary to use a longer or shorter cannula in order to correctly reach the semen deposition site.

Due to their functional characteristics, the catheters used tend to have a considerable length which always outwardly protrudes from the animal.

This fact entails a drawback in using catheters, since it involves working with animals and in the time period that lapses between the introduction of the catheter and the cannula, the animal may make sudden movements or sit down, such that the part of the catheter protruding from the animal can come into contact with dirt on the ground. In these cases and in order to prevent any infection in the animal when introducing the semen, the catheter must be removed and replaced with another one, whereby causing an economic loss and a time loss, the effectiveness of the process being reduced.

### DESCRIPTION OF THE INVENTION

The closure plug for a catheter presented herein comprises a hollow tubular main body with two open opposite ends, the first end of said ends being the coupling end for coupling to the final end of the catheter.

The main body has an inner diameter equal to the outer diameter of the catheter at said final end thereof.

The closure plug in turn comprises a tubular secondary body with a first open end for coupling through same to the second end of the main body and a second closed end opposite the first end.

Therefore, the coupling or uncoupling of the secondary body with respect to the main body causes the closing or opening of the main body and therefore of the catheter to which said main body is in turn coupled.

This closure plug can comprise securing means for securing the secondary body to the main body so that said secondary body will not be misplaced when being uncoupled.

The main body of the plug can also comprise a support flange for supporting the secondary body.

Said main body can also comprise coupling means for coupling to the final end of the catheter, likewise, the secondary body can also comprise coupling means for coupling to the second end of the main body.

This closure plug for a catheter has a main body made of a semi-flexible material, such that it allows certain flexibility in response to possible movements of the animal when applying the dose of semen. Particularly, both the main body and the secondary body are made of a plastic material.

One and the same mold can also be used for preparing said closure plug, which facilitates the process and favors obtaining the exact measurements for a perfect coupling between both bodies forming it.

In this case, where one and the same mold is used for preparing the main and secondary bodies, these bodies must be made of a plastic material each with a different melting point so that the melting of the second body does not alter the nature of the first body.

This closure plug for a catheter can also comprise gripping means in the secondary body to facilitate opening said body with respect to the main body.

A significant improvement with respect to the state of the art is achieved with the closure plug for a catheter proposed herein since the final end of the catheter is kept free from the possibility of coming into contact with dirt or contamination from the outside since they are closed and protected.

When connecting the bag of semen to the catheter, the secondary body of the plug is removed such that the connection thereof to the bag is in perfect hygiene and health conditions to be able to perform the insemination process without any risk for the animal.

In addition to the clear health and hygiene advantages, in turn a more effective insemination process which requires less time and lower costs is thereby achieved since it is not necessary to repeat the process due to accidental contamination.

### BRIEF DESCRIPTION OF THE DRAWINGS

For the purpose of aiding to better understand the features of the invention according to a preferred practical embodiment thereof, a set of drawings is attached as an integral part of said description in which the following has been depicted with an illustrative and non-limiting character:

FIG. 1 shows elevational and profile views of the closure plug for a catheter in a first closed position.

FIG. 2 shows a perspective view of the closure plug for a catheter in a second open position.

FIG. 3 shows a perspective view of the closure plug coupled to the final end of a catheter.

FIG. 4 shows a perspective view of the closure plug for a catheter in a first closed position, in which case it has a ribbed finish in the lower part of the main body.

### DETAILED DESCRIPTION OF A PREFERRED EMBODIMENT OF THE INVENTION

In view of the drawings provided, it can be observed how in a preferred embodiment of the invention, the closure plug 1 for a catheter 2 proposed herein comprises a main body 3 and a secondary body 4.

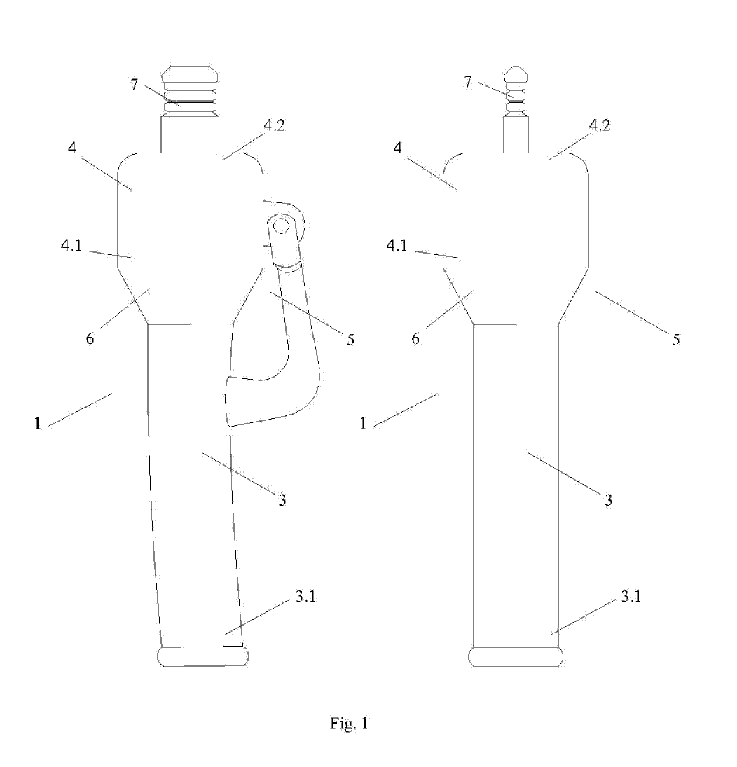

As can be observed in FIGS. 1 to 3, the main body 3 has a hollow tubular shape with two open opposite ends, where the first end 3.1 of said ends is the coupling end for coupling said main body 3 to the final end of the catheter 2. The inner diameter of this main body 3 is equal to the outer diameter of said final end of the catheter 2.

The secondary body 4 has a tubular shape with a first open coupling end 4.1 for coupling to the second end 3.2 of the main body 3, and a second closed end 4.2 opposite the first end 4.1.

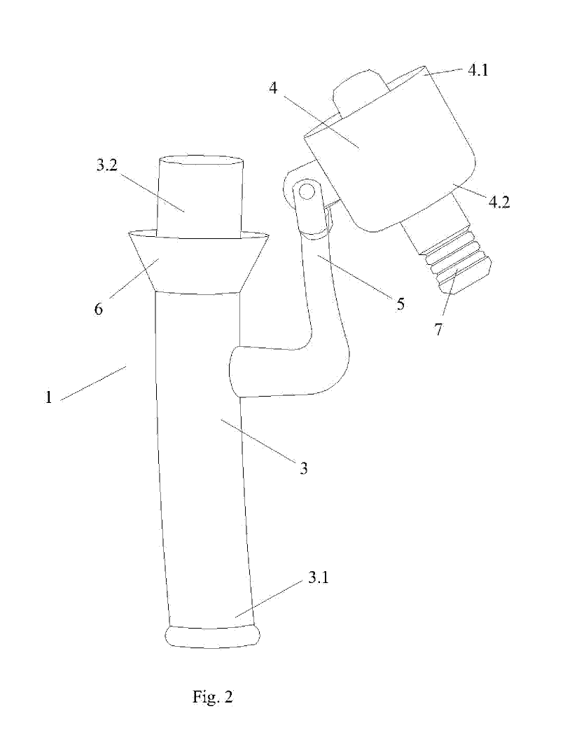

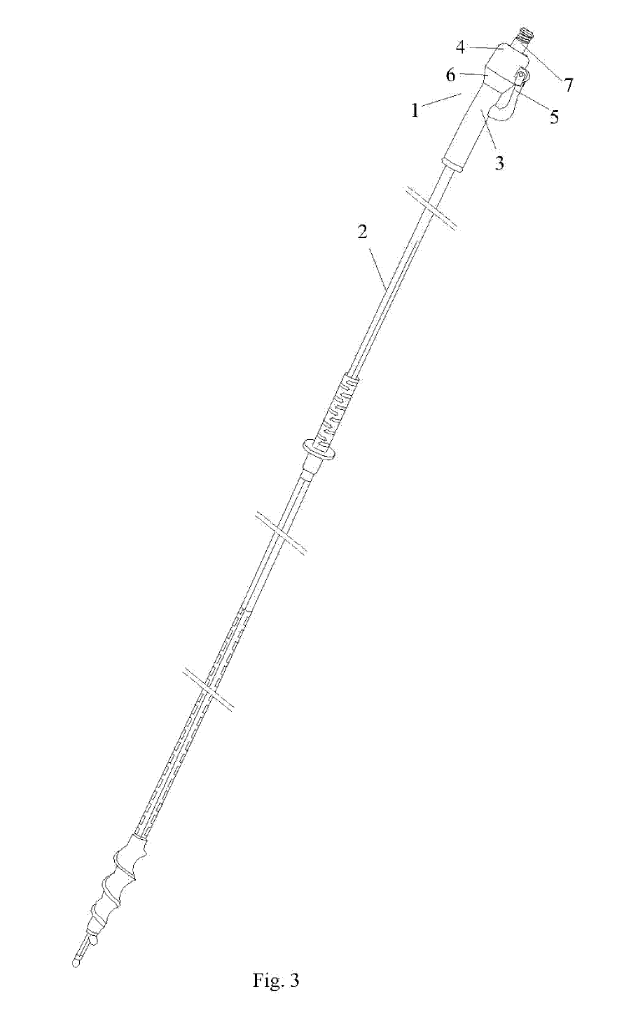

Therefore, when the secondary body 4 is coupled to the main body 3, as shown in FIG. 1, the plug 1 is closed. Since the main body 3 is in turn coupled to the final end of the catheter 2, as shown in FIG. 3, it causes the closing of said final end of the catheter 2, protecting it from external contamination, whereas when the secondary body 4 is removed from its coupling at the second end 3.2 of the main body 3, as observed in FIG. 2, the catheter 2 is open at its final end and in perfect conditions so that the semen containing bag is connected thereto.

In this preferred embodiment of the invention, the closure plug 1 comprises securing means 5 for securing the secondary body 4 to the main body 3 and said main body 3 has a support flange 6 for supporting the secondary body 4.

Likewise, both the coupling of the main body 3 to the final end of the catheter 2 and the coupling of the secondary body 4 to the second end 3.2 of the main body 3 are carried out by fitting the ends together, without needing to use additional coupling means in this preferred embodiment of the invention.

The main body 3 is made of a semi-flexible material to allow certain movement with respect to the catheter in response to the possible movement of the animal when injecting the semen.

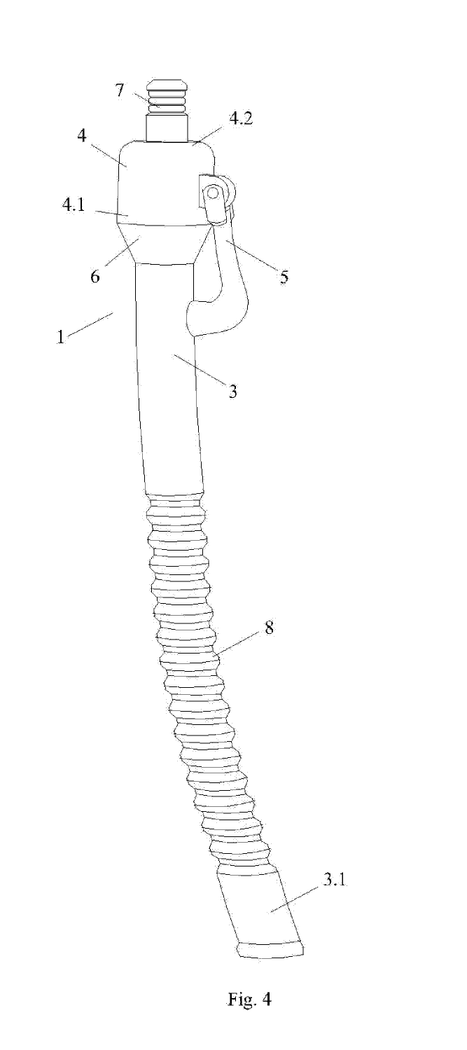

As a particular case of this preferred embodiment of the invention, FIG. 4 shows a second example of a closure plug 1, similar the preceding plug, but in which the main body 3 comprises a ribbed finish 8 in a band following its first end 3.1 to improve the flexibility of said main body 3 in this area, thus providing the conditions when injecting the semen into the animal.

In this preferred embodiment of the invention, both this main body 3 and the secondary body 4 are made of a plastic material, and in this case, they are prepared with one and the same mold, for which purpose the plastic material for forming each body has a different melting point, such that the melting of the first body does not alter the nature of the second body.

This closure plug for a catheter in turn comprises gripping means 7 at the second end 4.2 of the secondary body 4 to thus facilitate opening said body with respect to the main body 3.

The state of the art is significantly improved with the closure plug for a catheter presented herein.

This is because a drawback causing an increase in costs and times used in the insemination process is solved by using this closure plug for a catheter.

Therefore, the use of this plug keeps the final end of the catheter protected against possible contamination, thereby allowing a much quicker, safer, more effective and more economical process since the need to repeat the process in the event of a possible contamination of the final end of the cannula are eliminated.

1. A closure plug for a catheter, comprising a hollow tubular main body with two open opposite ends, the first end of said ends being the coupling end for coupling to the final end of the catheter, where the main body has an inner diameter equal to the outer diameter of the catheter at said final end thereof, and a tubular secondary body with a first open coupling end for coupling to the second end of the main body and a second closed end opposite the first end.

2. The closure plug for a catheter according to claim 1, further including securing means for securing the secondary body to the main body.

3. The closure plug for a catheter according to any of the claim 1, wherein the main body comprises a support flange for supporting the secondary body.

4. The closure plug for a catheter according to claim 1, wherein the main body comprises coupling means for coupling to the final end of the catheter.

5. The closure plug for a catheter according to claim 1, wherein the secondary body comprises coupling means for coupling to the second end of the main body.

6. The closure plug for a catheter according to claim 1, wherein the main body is made of a semi-flexible material.

7. The closure plug for a catheter according to claim 1, wherein the main body and the secondary body are made of a plastic material.

8. The closure plug for a catheter according to claim 1, wherein the main body and the secondary body are made with one and the same mold.

9. The closure plug for a catheter according to claim 7, wherein the main body and the secondary body are made of a plastic material with a different melting point.

10. The closure plug for a catheter according to claim 1, wherein the secondary body comprises gripping means.
